(19) United States
(12) Patent Application Publication
(10) Pub. No.: US 2008/0305519 A1
(43) Pub. Date: Dec. 11, 2008

(54) Biochemical method for specific protein labeling

(76) Inventors: Qing Lin, Getzville, NY (US); Michael M. Madden, Buffalo, NY (US)

Correspondence Address: PHILLIPS LYTLE LLP;INTELLECTUAL PROPERTY GROUP, 3400 HSBC CENTER, BUFFALO, NY, 14203-3509, US

(21) Appl. No.: 11/709,949

(22) Filed: Feb. 23, 2007

Related U.S. Application Data
(60) Provisional application No. 60/776,118, filed on Feb. 23, 2006.

(51) Int. Cl. C12P 21/06; C07K 7/00; C07D 235/02

(52) U.S. Cl. 435/68.1; 530/300; 548/304.1; 530/329; 530/330; 530/331

(57) ABSTRACT

An improved method for protein labeling comprising the steps of providing a synthetic small molecule tag, providing a target protein to be tagged, providing at least two enzymes for catalyzing a conjugation reaction between the tag and the target protein, incubating the tag, the protein and the enzyme, and allowing the tag to conjugate to the target protein. The tag may embody at least one structural feature of an ubiquitin C-terminus, and the structural feature may comprise a recognition sequence that is recognizable by an ubiquitin activating enzyme.

### CROSS-REFERENCE TO RELATED APPLICATIONS

This application claims the benefit of U.S. Provisional Patent Application No. 60/776,118, filed Feb. 23, 2006. The entire content of such application is incorporated by reference herein.

### TECHNICAL FIELD

The present invention relates generally to protein labeling and, more particularly, to a method of direct transfer of synthetic small-molecule tags onto proteins by the ubiquitination pathway.

### BACKGROUND ART

Selective labeling of proteins with novel chemical tags is a powerful strategy for the study of protein structure, dynamics and function (Yang et al., Science 1990, 249, 1398-405; Lu et al., Nat Neurosci 2001, 4, 239-46; Gygi et al., Nat Biotechnol 1999, 17, 994-9; Griffin et al., Science 1998, 281, 269-72; and Chen et al., Curr Opin Biotechnol 2005, 16, 35-40). Strategies based on the chemical, chemoenzymatic, and biosynthetic-pathway approaches have been developed to incorporate unique chemical tags onto select amino acid side chains in a protein of interest (Prescher et al., Nature 2004, 430, 873-7; Saghatelian et al., Proc Natl Acad Sci USA 2004, 101, 10000-5; and Clarke et al., J Am Chem Soc 2005, 127, 11234-5). However, a general strategy that can apply to unmodified, native proteins across a wide spectrum of protein families is still lacking.

Direct chemical modification through side-chain specific reactions proves to be a versatile method in labeling protein with desired chemical functionality. By exploiting unique chemical reactivity of each side-chain, a wide range of mild, residue-specific chemistries have been developed for cysteines (Levine et al., J. Am. Chem. Soc. 1978, 100, 7670-7677; and Kaiser et al., Science 1984, 226, 505-11), lysines (McFarland et al., J Am Chem Soc 2005, 127, 13490-1), tyrosines (Tilley et al., J Am Chem Soc 2006, 128, 1080-1; Joshi et al., J Am Chem Soc 2004, 126, 15942-3), and tryptophans (Antos et al., J Am Chem Soc 2004, 126, 10256-7). Chemical approach allows rapid access of unlimited small-molecule moieties, but does not confer selectivity among identical amino acid residues commonly present in a protein target. To overcome this limitation, ligation-based methods, such as enzyme-assisted ligation (Jackson et al., Science 1994, 266, 243-7), chemical ligation (Schnolzer et al., Science 1992, 256, 221-5; Low et al., J. Am. Chem. Soc. 1998, 120, 11536-11537; and Kochendoerfer et al., Science 2003, 299, 884-7), and expressed protein ligation (Muir et al., Proc Natl Acad Sci USA 1998, 95, 6705-10; Cotton et al., J. Am. Chem. Soc. 1999, 121, 1100-1101; and Arnold et al., J. Am. Chem. Soc. 2002, 124, 8522-8523), have been successfully developed in which large proteins are assembled from small protein fragments containing synthetic moieties.

In contrast, the chemoenzymatic approach offers high labeling selectivity and, in most cases, versatility for in vivo protein modification in living cells. It relies upon either promiscuous substrate specificity of native enzymes or altered specificity in the engineered enzymatic systems, e.g. the use of ketone derivatives of GalNAc-UDP by an engineered β-1,4-galactosyltransferase (Khidekel et al., J Am Chem Soc 2003, 125, 16162-3; and Tai et al., J Am Chem Soc 2004, 126, 10500-1), the use of a biotin mimic by biotin ligase BirA (Chen et al., Nat Methods 2005, 2, 99-104; and Howarth et al., Proc Natl Acad Sci USA 2005, 102, 7583-8), the use of cadaverin derivatives by transglutaminase (Lin et al., J Am Chem Soc 2006, 128, 4542-3), the use of CoA derivatives by phosphorpantetheine transferase (Clarke et al., J Am chem. Soc 2005, 127, 11234-5; Vivero-Pol et al., J Am Chem Soc 2005, 127, 12770-1; and La Clair et al., Chem Biol 2004, 11, 195-201), and the use of O⁶-benzylguanine derivatives by DNA alkyltransferase (Keppler et al., Nat Biotechnol 2003, 21, 86-9; Gendreizig et al., J Am Chem Soc 2003, 125, 14970-1; Juillerat et al., Chem Biol 2003, 10, 313-7; Keppler et al., Methods 2004, 32, 437-44; and Keppler et al., Proc Natl Acad Sci USA 2004, 101, 9955-9). Small-molecule tagging selectivity is achieved by fusing either a substrate domain or the enzyme itself directly to protein targets.

The protein biosynthetic pathway has also been exploited to incorporate unnatural amino acids into proteins with exquisite site-specificity (Xie et al., Curr Opin Chem Biol 2005, 9, 548-54; and Wang et al., Angew. Chem. Int. Ed. 2005, 44, 34-66). A diverse set of small-molecule probes has been successfully added into the genetic code of prokaryotes and eukaryotes (Wang et al., Science 2001, 292, 498-500; Chin et al., Science 2003, 301, 964-7; Alfonta et al., J Am Chem Soc 2003, 125, 14662-3; Zhang et al., Science 2004, 303, 371-3; Xie et al., Nat Biotechnol 2004, 22, 1297-301; Wu et al., J Am Chem Soc 2004, 126, 14306-7; Xu et al., J Am Chem Soc 2004, 126, 15654-5; Deiters et al., Bioorg Med Chem Lett 2005, 15, 1521-4; Bose et al., J Am Chem Soc 2006, 128, 388-9; Summerer et al., Proc Natl Acad Sci USA 2006, 103, 9785-9; Tsao et al., J Am Chem Soc 2006, 128, 4572-3; Ryu et al., Nat Methods 2006, 3, 263-5; and Deiters et al., Angew Chem Int Ed Engl 2006, 45, 2728-31). Incorporation specificity was achieved by placing an Amber stop codon TAG at the desired location in the protein-encoding DNA. Similarly, synthetic unnatural sugars have been introduced into the glycan structures on glycoproteins through the polysaccharide metabolic pathway (Prescher et al., Nature 2004, 430, 873-7; Mahal et al., Science 1997, 276, 1125-8; and Bertozzi et al., Science 2001, 291, 2357-64). However, this approach can only be applied to glycoproteins; it remains to be established how one particular glycoprotein can be selectively tagged in the presence of thousands of other potential targets.

The ubiquitination pathway offers a powerful biochemical mechanism for protein posttranslational modification because: 1) ubiquitin is a universal protein modifier regulating the fate of the majority of intracellular proteins through the proteasome-dependent proteolysis (Ciechanover, EMBO J. 1998, 17, 7151-7160 and Peng et al., Nat Biotechnol 2003, 21, 921-6); 2) the pathway operates through a modular enzymatic cascade involving successive actions of three distinct enzymes: an activating enzyme E1, a conjugating enzyme E2 and a ligase E3 (Pickart, Annu. Rev. Biochem. 2001, 70, 503-533); c) a vast pool of transfer enzymes including ˜30 conjugation enzymes (E2s) and greater than 500 ligases (E3s) are encoded in the human genome to regulate temporospatial expression of selected substrates (Semple, Genome Res 2003, 12, 1389-94); d) roughly 15 ubiquitin-like modifiers (Ubls) have been identified in eukaryotic genomes that share the same mechanism of activation and transfer (Hochstrasser, Science 2000, 289, 563-4; and Schwartz et al., Trends Biochem Sci 2003, 28, 321-8), suggesting that this activation-conjugation-ligation modality is highly conserved during evolution. Based on these characteristics, Applicants have developed a method in which the modality of the ubiquitination pathway is harnessed to transfer small-molecule tags to selected protein targets. Targeting specificity can be achieved by a temporal expression of particular E3 ligases in conjunction with the exertion of appropriate ubiquitination signals.

### DISCLOSURE OF THE INVENTION

With parenthetical reference to the corresponding parts, portions, steps or surfaces of the disclosed embodiment, merely for the purposes of illustration and not by way of limitation, the present invention broadly provides an improved method for labeling a protein comprising the steps of providing a synthetic small molecule tag, providing a target protein to be tagged, providing at least two enzymes for catalyzing a conjugation reaction between the tag and the target protein, incubating the tag, the protein, and the enzyme, and allowing the tag to conjugate to the target protein.

The tag may embody at least one structural feature of an ubiquitin C-terminus, and the structural feature may comprise a recognition sequence that is recognizable by an ubiquitin activating enzyme. The tag may comprise a probe and a recognition sequence. The probe may comprise biotin or a fluorophore, and the recognition sequence may be recognizable by an ubiquitin activating enzyme. The probe and the recognition sequence may be linked by a flexible aminohexanoic acid linker.

The protein may be a substrate for an ubiquitin conjugation system, e.g. tubulin, or protein mixtures in Fraction II of a reticulocyte lysate, or a ligase E3-specific substrate.

The enzymes may be selected from a family of ubiquitin conjugating enzymes. The enzymes may be an ubiquitin activating enzyme and an ubiquitin conjugating enzyme, and the method may further comprise the steps of providing an ubiquitin ligase enzyme, incubating the tag, the protein, the activating enzyme and the conjugating enzyme, or incubating the tag, the protein, the activating enzyme, the conjugating enzyme and the ligase enzyme.

The step of incubating the tag, the protein and the enzymes may comprise the step of applying an ATP-supplemented reaction buffer and may comprise the step of incubating the mixture at 37° C.

The present invention also provides a compound for tagging a protein comprising a probe and a recognition sequence that is recognizable by an ubiquitin activating enzyme.

The recognition sequence may be a peptide sequence derived from an ubiquitin C-terminus.

The probe may comprise biotin, a fluorophore, and other types of small-molecule biophysical probes. The probe and the recognition sequence may be linked by a flexible linker such as aminohexanoic acid.

The recognition sequence may comprise a peptide sequence selected from the group consisting of Leu-Arg-Leu-Arg-Gly-Gly (SEQ ID NO: 1), Leu-Ala-Leu-Arg-Gly-Gly (SEQ ID NO: 2), Arg-Leu-Arg-Gly-Gly (SEQ ID NO: 3), Leu-Arg-Gly-Gly (SEQ ID NO: 4), Arg-Gly-Gly (SEQ ID NO: 5), and Gly-Gly (SEQ ID NO: 6).

These and other objects and aspects will become more apparent from the foregoing and ongoing written specification, the drawings, and the appended claims.

The term “recognition sequence” as used herein denotes a sequence of amino acids recognizing, or exhibiting binding specificity for, a known enzyme or peptide or other binding partner.

The term “synthetic” as used herein means derived from chemical synthesis and not by biological recombination.

The term “incubating” as used herein refers to the act of placing two reagents in such relationship that they may interact in order to produce a chemical or biological effect. The process may include mixing the reagents in an appropriate buffer.

The term “probe” as used herein refers to the label (radioactive, antigen, molecular enzyme, fluorescent) that, with a recognition sequence, is used to facilitate functional annotation of a protein of interest after incubating with the protein target.

### BRIEF DESCRIPTION OF THE DRAWINGS

FIG. 1 shows small-molecule tag transfer by the ubiquitination pathway in a cell-free system. The biotin-labeled proteins are probed with streptavidin-alkaline phosphatase and visualized with chemiluminescence. (a) Schematic diagram of a small-molecule tag transfer pathway mediated by the ubiquitination enzymes E1, E2 and E3; (b) The formation of E1-1 thioester intermediates; (c) The transfer of tag 1 from E1-1 to E2 to form the E2-1 thioesters; and (d) The formation of protein-1 adducts in the fraction II of the rabbit reticulocyte lysate.

FIG. 2 shows selective labeling of tubulin by parkin: (a) Concentration-dependent labeling of tubulin by tag 1; and (b) Structure-labeling efficiency study with the small-molecule tags 1-6 carrying various lengths of recognition sequence.

FIG. 3 is a series of small-molecule tags (1-6) synthesized by linking a biotin molecule to varying lengths of the recognition sequences through a flexible aminohexanoic acid linker.

FIG. 4 is the mass spectrum characterization of the synthetic biotin tags.

FIG. 5 shows the two functional domains of an ubiquitin molecule (PDB code: 1UBQ). The chemistry domain comprising of the C-terminal recognition sequence LRLRGG is shown in green tube model while the effector binding domain comprising of the globular region is shown in blue wire model.

FIG. 6 demonstrates the E1-1 adduct formation is ATP-dependent with transfer efficiency critically dependent on the recognition sequence LRLRGG: the incubation of E1 with tag 1 (150 μM) in the presence of 10 μM ATP (lane 1) or absence of ATP (lane 2), or with tag 2 at concentrations of 450 μM (lane 3), 150 μM (lane 4), 50 μM (lane 5), respectively.

FIG. 7 is a biotinylated ubiquitin (Bio-Ub) tag transfer catalyzed by the ubiquitination enzymes E1 and E2: lane 1, the biotinylated ubiquitin input, notice the presence of a minor dimeric ubiquitin component; lane 2, tag 1 transfers to E1 and E2 to form E1-1 (*) and E2-1 (#) adducts, respectively; lane 3, E1 forms an adduct with Bio-Ub with a corresponding increase in molecular weight (MW) due to the addition of 8.5 KD ubiquitin; lane 4, incubation of Bio-Ub with E2 alone does not lead to the formation of E2-Ub adduct; lane 5-7, incubation of Bio-Ub with both E1 and E2 leads to the concentration-dependent formation of the E1-Ub and E2-Ub adducts, notice the MW increases in the E2-Ub adducts; lane 8-10, as concentrations of Bio-Ub dropped below 21 nM, no Ub-adducts were observed; lane 11, the addition of 300 mM DTT after the Ub-E1-E2 reaction caused the disappearance of the E1-Ub and E2-Ub adduct bands, suggesting the linkages to be thioester bonds.

FIG. 8 shows that the tubulin modification by tag 1 is mediated through ubiquitination pathway involving the thioester intermediates formed between the enzymes and the recognition sequence LRLRGG present in the tag structure. Robust labeling was observed when tag 1 was incubated with the tubulin/parkin complex (lane 3). The adduct was DTT resistant as the reaction mixture was boiled in SDS sample buffer containing 100 mM DTT at 95° C. for 5 minutes. The labeling was abolished when the tubulin/parkin complex was heat-inactivated with SDS buffer containing 20 mM mercaptoethanol (lane 4) or pre-treated with 600 mM DTT (lane 5). The labeling requires the LRLRGG recognition sequence as the long-chain biotin molecule (LC-biotin, dissolved in DMSO then diluted into the reaction buffer) itself could not label the tubulins (lane 6) while the same amount of DMSO content (5%) in the reaction buffer did not affect the tag 1 transfer by the ubiquitination enzymes present in the tubulin/parkin complex.

### DESCRIPTION OF RECOGNITION SEQUENCES FOR THE PREFERRED EMBODIMENTS

SEQ ID NO: 1 is the peptide sequence Leu1 Arg2 Leu3 Arg4 Gly5 Gly6.

SEQ ID NO: 2 is the peptide sequence Leu1 Ala2 Leu3 Arg4 Gly5 Gly6.

SEQ ID NO: 3 is the peptide sequence Arg1 Leu2 Arg3 Gly4 Gly5.

SEQ ID NO: 4 is the peptide sequence Leu1 Arg2 Gly3 Gly4.

SEQ ID NO: 5 is the peptide sequence Arg1 Gly2 Gly3.

SEQ ID NO: 6 is the peptide sequence Gly1 Gly2.

### DESCRIPTION OF THE PREFERRED EMBODIMENTS

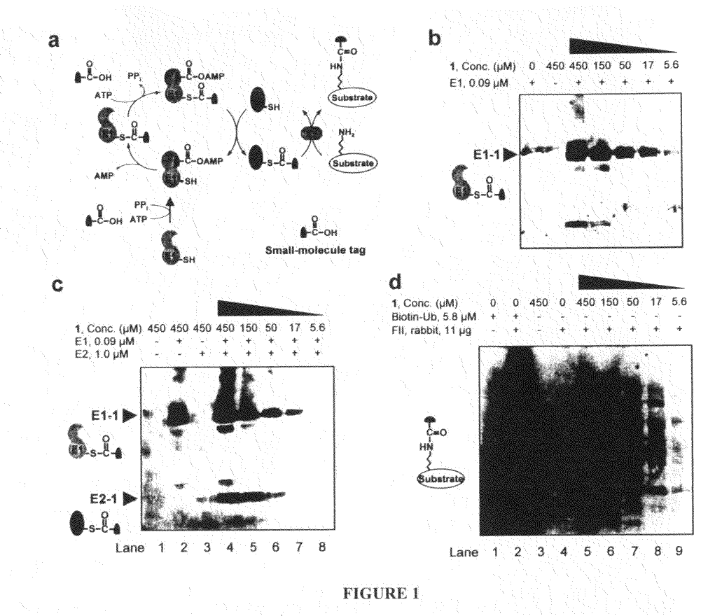

An important lesson can be learned from Nature on how protein posttranslational modifications are carried out in living cells. While most cellular protein modifications, such as phosphorylation, acetylation, glycosylation, methylation, and nitrosylation, are carried out by specific classes of enzymes, protein ubiquitination appears to be a more wide-spread posttranslational modification in eukaryotic proteomes, e.g., 1075 ubiquitinated proteins were identified among the 6139-membered yeast proteome (Peng et al., Nat Biotechnol 2003, 21, 921-6). In the ubiquitination pathway, ubiquitin is conjugated to the target protein surface lysines via isopeptide linkage through an enzymatic cascade involving successive action of three enzymes: an activating enzyme E1, a conjugating enzyme E2, and a ligase E3. Targeting specificity of the pathway is achieved primarily through selective recruitment of target proteins by hundreds of distinct E3 ligases (Pickart, Annu. Rev. Biochem. 2001, 70, 503-533). As shown in FIG. 1, this endogenous biochemical pathway can be utilized to transfer small-molecule tags that mimic the structure of ubiquitin directly onto E3-specific protein substrates in a cell-free system, with the transfer efficiency critically dependent on the recognition sequence.

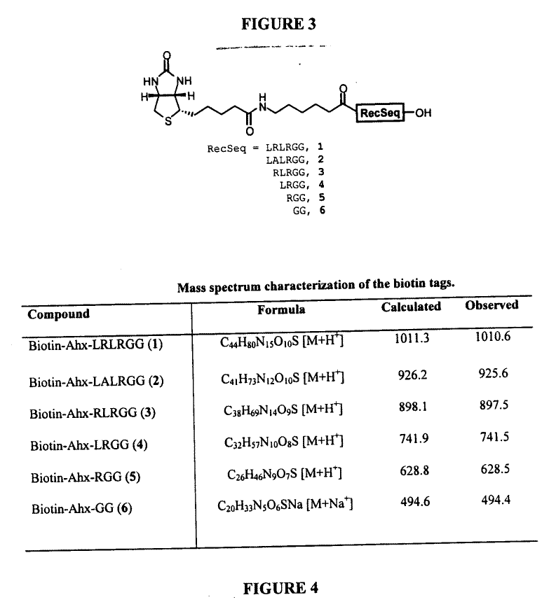

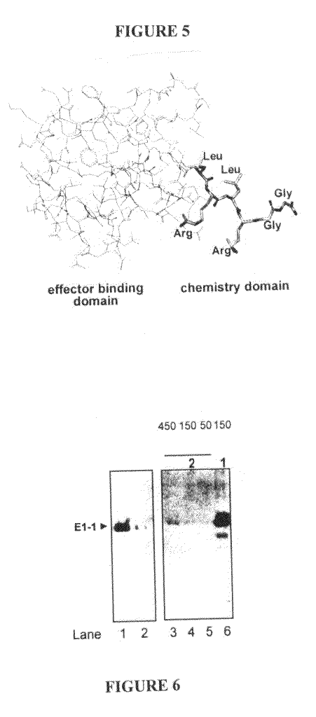

As shown in FIG. 5, a number of studies including mutagenesis, biochemical characterization, and structural analysis suggest that ubiquitin can be divided into two functional domains: a chemistry domain encompassing the C-terminal tail region responsible for the ubiquitin chain-transfer reactions and an effector-binding domain composed of the globular region recognizable by diverse ubiquitin interacting partners (Sloper-Mould et al., J. Biol. Chem. 2001, 276, 30483-30489; Miura et al., J. Mol. Biol. 1999, 290, 213-228; and Hamilton et al., Structure 2001, 9, 897-904). Furthermore, a synthetic C-terminal fragment of ubiquitin was reported to stimulate the pyrophosphate-ATP exchange, the first step during ubiquitin activation by E1 enzyme (Jonnalagadda et al., J. Biol. Chem. 1988, 263, 5016-5019). Accordingly, the ubiquitin C-terminus can serve as a delivery vehicle for small-molecule tags targeting the protein surface lysines via the ubiquitination pathway, and targeting specificity can be achieved by expression of specific E3 ligases which mediate the rate-limiting step of the entire pathway. Thus, as shown in FIG. 3, a series of small-molecule tags 1-6 were synthesized by linking a biotin molecule to varying lengths of the ubiquitin C-terminal recognition sequences through a flexible aminohexanoic acid linker.

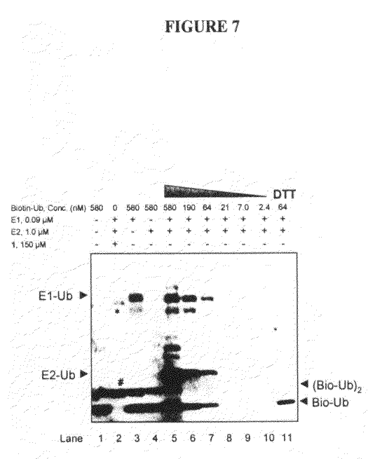

To assess the biotin tag transfer along the ubiquitination pathway, the formation of the E1-1 and E2-1 thioester intermediates, shown in FIG. 1a, was probed in a cell-free, reconstituted model with the purified E1 and E2 enzymes. As shown in FIG. 1b, the biotin-containing protein bands with the size matching that of E1 were detected after incubating tag 1 with E1 for 5 min in a non-reducing, ATP-supplemented reaction buffer. Lowering the concentrations of tag 1 led to proportional reduction in the E1-biotin adducts, in agreement with the ubiquitin activating mechanism under the single turnover condition (Haas et al., J. Biol. Chem. 1982, 257, 10329-10337). The E1-1 adduct was labile to the 1,4-dithiothreitol (DTT) treatment, indicating that the linkage is through the thioester bond. In addition, as indicated in FIG. 6, withdrawal of ATP from the reaction buffer aborted the biotin-adduct formation, indicating the tag transfer to E1 is ATP-dependent. With further reference to FIG. 6, the recognition sequence, LRLRGG (SEQ ID NO: 1), of tag 1 was found to be very critical as an analogous compound (2) with the sequence, LALRGG (SEQ ID NO: 2), showed almost no activity at concentrations as high as 450 μM. Significantly, as indicated in FIG. 1c (lane 4 and 5), addition of E2 into the E1-1 adduct led to the appearance of a second biotin-containing band with the size matching that of E2, suggesting the tag 1 transfer from the E1-1 intermediate onto the E2 enzyme. It is noteworthy that as indicated in FIG. 1c (lane 3) that the incubation of tag 1 with E2 in the absence of E1 did not give rise to the biotin-labeled E2, which indicates the tag transfer is mediated by E1, likely through the E1-1 thioester intermediate. When tag 1 concentration was reduced to 50 μM, the extent of tag transfer from E1-1 to E2 diminished to undetectable level. Both E1-1 and E2-1 intermediates bands were susceptible to the DTT treatment, in agreement with the existence of the thioester linkage (data not shown). By comparison, as indicated in FIG. 7, in a parallel assay using a biotinylated ubiquitin as the tag, the formation of the ubiquitin-thioester intermediates were observed similarly, albeit with ca. 265-fold higher efficiency as the ubiquitin activation was detectable at the ubiquitin concentrations as low as 64 nM. Furthermore, as shown in FIG. 1d, in a classic ubiquitination assay using the ubiquitin-free reticulocyte fraction II cell extract which contains E1, E2, and E3 activities (Ciechanover et al., Proc Natl Acad Sci USA 1980, 77, 1365-1368), supplementation of tag 1 led to concentration-dependent covalent modification of a large number of proteins in the extract. These tag 1-derived adducts are robust as the treatment of 100 mM DTT after the incubation did not reduce their intensities on the SDS-PAGE gel, further confirming the presence of isopeptide linkages between tag 1 and the modified proteins (data not shown). By comparison, as indicated in FIG. 1d (lane 2), the incubation with the biotin-ubiquitin tag entailed massive protein ubiquitination in the same extract, consistent with the observed higher transfer efficiency for the full-length ubiquitin.

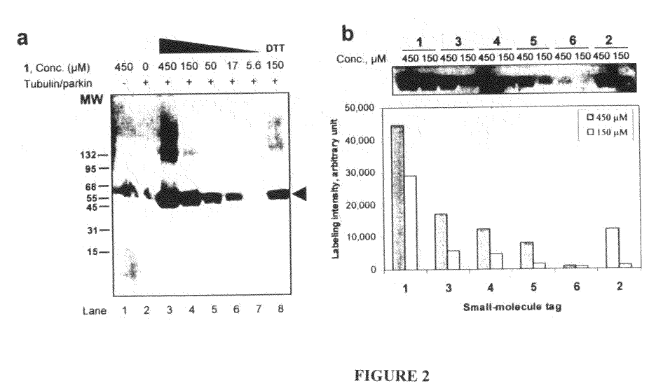

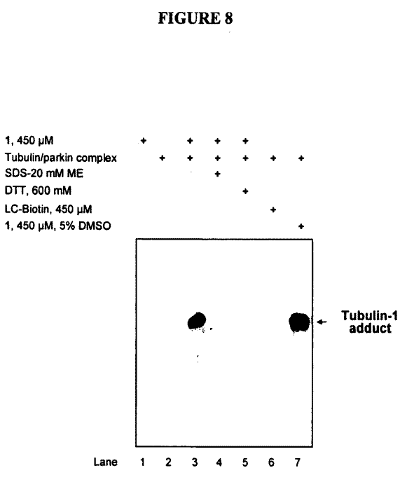

To confirm that small-molecule tags can be selectively transferred onto protein substrates as selected by an E3 ligase, a semi-purified rat brain tubulin/parkin complex, which contains endogenous E1 and E2 activities and is enriched in parkin, an E3 ligase, and its substrate, tubulin (Ren et al., J. Neurosci. 2003, 23, 3316-3324), was incubated with tag 1 in a reaction mixture containing 10 μM ATP. As shown in FIG. 2a, concentration-dependent biotin labeling of a 55 KDa protein was observed in a concentration range of 17-450 μM, which bears striking resemblance to the concentration-dependent labeling of E1 shown in FIG. 16 (FIG. 1b) as well as Fraction II in FIG. 1d. With reference to FIG. 2a, the identity of the labeled bands was confirmed to be tubulin both by immunoblotting with anti-α-tubulin antibody and by their ability to re-assemble to produce high MW tubulin oligomers during prolonged incubation at 37° C. (weaker upper bands at lane 3) which can also be completely abolished in the presence of 10 μM colchicine, a microtubule assembly inhibitor (data not shown). With reference to FIG. 2a (lane 8), the labeling of tubulin appears to mediate through isopeptide linkage as the biotin-adduct was resistant to the DTT treatment. As shown in FIG. 8, pre-incubating the tubulin/parkin complex with either mercaptoethanol in the SDS sample buffer with heating or treatment with 600 mM DTT abolished the labeling, indicating that the ubiquitination pathway was directly involved. Again with reference to FIG. 8, the incubation of the semi-purified tubulin/parkin complex with the long-chain biotin (biotinylaminohexanonic acid) at 450 μM concentration caused no labeling of any protein in the reaction mixture, indicating the recognition motif within tag 1 is absolutely required. As shown in FIG. 2b, the transfer efficiency of this parkin-mediated tubulin modification depends critically on the recognition sequence as both the substitution (2) and the gradual shortening of the recognition sequence (3-6) (SEQ ID NO: 3-6) resulted in the decreased biotin labeling. The LRLRGG sequence afforded the highest labeling efficacy, presumably due to a tighter binding between tag 1 and E1 over much larger surface contact and thus more efficient E1-tag thioester formation (Walden et al., Mol Cell 2003, 12, 1427-37).

Thus, small-molecule tags containing the C-terminal fragments of ubiquitin are effectively conjugated to the ubiquitination enzymes E1 and E2 in a purified enzymatic system, and successively transferred onto protein substrates in a reticulocyte lysate fraction. The specific labeling of tubulin by biotin-derived tags was also observed in a semi-purified tubulin/parkin complex isolated from rat brains. Among other things, this pathway-enabled selective biotinylation of ubiquitin substrates can serve as useful proteomic tools for identifying protein substrates for various E3 enzymes in the complex ubiquitination pathway (Denison et al., Curr. Opin. Chem. Biol. 2005, 9, 69-75). Accordingly, biotin-derived synthetic small molecule tags carrying the ubiquitin C-terminal recognition sequence are adopted by the ubiquitination pathway and transferred directly onto the protein substrates in a cell-free system.

In the preferred embodiment, all amino acids, coupling reagents, resins, and solvents were purchased from commercial sources. The biotin tags were purified on a Gilson reverse phase HPLC system equipped with a Vydac 218TP1022 C18 column running a gradient of 10%-90% acetonitrile/0.1% TFA water over 30 min. The MS data were acquired from a Finnigan LCQ mass spectrometer. All purified ubiquitin enzymes and substrates were purchased from Boston Biochem (Cambridge, Mass.), including ubiquitin activating enzyme (E1), rabbit (cat. # E-302); UbCH7 (E2), human recombinant (cat. # E2-640); His₆-Biotin-N-terminal Ubiquitin, human recombinant (cat. # U-560); Fraction II, rabbit reticulocyte (cat. #F-360). The monoclonal anti-α-tubulin (clone DM1A) was purchased from Sigma (St. Louis, Mo.). The VECTASTAIN ABC-AmP system was purchased from Vector Labs (Burlingame, Calif.). The Tris-glycine precast SDS-PAGE gels and the PVDF membrane were purchased from Invitrogen (Carlsbad, Calif.). The semi-purified parkin-tubulin complex from rat brain homogenates was obtained by washing taxol-assembled microtubules with 2M NaCl as described previously (Yang et al. J. Biol. Chem. 2005; 280, 17154-17162).

### Synthesis of Biotin Tags

The compounds were synthesized in a plastic reaction vessel equipped with polymeric filtration frits, starting from the preloaded Fmoc-Gly-Wang resin (Bachem, Pa.). The standard Fmoc peptide coupling procedures were followed, i.e. 3 eq. Fmoc-AA-OH, 3 eq. HBTU, 6 eq. DIEA and appropriate amount of DMF to make 150 mM coupling solution. The elongated peptide was finally capped with 3 eq. of biotin under the same coupling condition. The biotin-modified peptide was cleaved from the resin with a TFA cleavage cocktail containing 2.5% TIS and 2.5% H₂O, and precipitated out with ethyl ether. The residue was dried and applied to a preparative reverse-phase HPLC running 10-90% ACN/H₂O gradient with a 20 mL/min flow rate. The fractions were checked by LC-MS and analytic HPLC, and the correct fractions were pooled and lyophilized to afford the titled compound in powder form.

### E1-Biotin Tag Conjugation Assay

The E1 enzyme was diluted with 50 mM HEPES buffer, pH 7.6 to derive the 0.45 μM stocks. A total of 90 nM rabbit E1 and varying concentrations of tag 1 was incubated at 37° C. for 5 min in 10 μL buffers of 50 mM Tris, 50 mM NaCl, 10 mM MgCl₂, 10 μM ATP, pH 7.6. The reactions were terminated by the additions of 2 μL 6× bromophenol-absent SDS sample buffer and the mixtures were boiled at 85° C. for 2 min. The biotin-labeled E1 thioesters were resolved from the free biotin tags with 8-16% Tris-Glycine SDS-PAGE gel, transferred onto the PVDF membrane using a semi-dry protein transfer apparatus, and detected using the VECTASTAIN ABC-AmP detection system following the manufacturer's protocol. Briefly, the membrane was washed three times for 4 min each in 10 mL 1× casein solution at r.t. with gentle shaking, and then incubated in the 10 mL 1× casein blotting solution containing 20 μL each of reagent A and B from the kit for 10 min. The membrane was washed three times for 4 min each in 10 mL 1× casein solution, followed by equilibration in 10 mL 0.1M Tris buffer, pH 9.5, for 5 min. The excess buffer was removed and the blot surface was incubated in 3 mL DuoLux chemiluminescent substrate for 5 min under subdued light. Briefly rinse the blot in 0.1 M Tris buffer, pH 9.5, and remove the excess buffer by touching the edge of the blot to absorbent paper. The image was obtained by exposing the blot to the Kodak BioMax Light Film.

### E2-Biotin Tag Conjugation Assay

The UbCH7 enzyme was diluted with a buffer of 50 mM HEPES, 50 mM NaCl, 10% glycerol, pH 7.6 to derive the 10 μM stocks. A solution of 90 nM rabbit E1, 1 μM UbCH7, and varying concentrations of tag 1 in 10 μL reaction buffer (50 mM Tris, 50 mM NaCl, 10 mM MgCl₂, 10 μM ATP, pH 7.6) was incubated at 37° C. for 5 min. Reactions were terminated by the additions of 2 μL 6× non-reducing bromophenol-absent SDS-PAGE sample buffer and the mixtures were boiled at 85° C. for 2 min. The biotin-labeled E1 thioesters were resolved from the free biotin tags with 8-16% Tris-Glycine SDS-PAGE gel, transferred onto the PVDF membrane using a semi-dry protein transfer apparatus, and detected using the VECTASTAIN ABC-AmP detection system following the manufacturer's protocol.

### Fraction II—Biotin Tag Conjugation Assay

The fraction II from rabbit reticulocyte lysates was cleaned with ImmunoPure immobilized streptavidin beads from Pierce (Rockford, Ill.) to remove non-specific streptavidin-binding proteins following the manufacturer's instruction. An aliquot (11 μg) was then incubated with various amounts of biotin tag in 10 μL reaction buffer (100 mM Tris, 3 mM DTT, 5 mM MgCl₂, 2 mM ATP, pH 7.6) at 37° C. for 5 min. The reactions were terminated by adding 2 μL 6×SDS-PAGE sample buffer and the mixtures were boiled at 85° C. for 2 min. The biotin-labeled substrates were resolved from the free biotin tags with 8-16% Tris-Glycine SDS-PAGE gel, transferred onto the PVDF membrane using a semi-dry protein transfer apparatus, and detected using the VECTASTAIN ABC-AmP detection system following the manufacturer's protocol.

### Biotin Tag Conjugation of Tubulin/Parkin Complex

A total of 4 μg semi-purified tubulin/parkin complex and varying concentrations of the biotin tag was incubated at 37° C. for 15 min in 10 μL buffers of 50 mM Tris, 50 mM NaCl, 10 mM MgCl₂, 10 μM ATP, pH 7.6. For DTT reduction, 1 μL of 6M DTT was added and the solution was incubated at r.t. for additional 30 min. Reactions were terminated by the additions of 2 μL 6×SDS-PAGE sample buffer containing 100 mM DTT and the mixtures were boiled at 95° C. for 5 min. The biotin-labeled substrates were resolved from the free biotin tags with 8-16% Tris-Glycine SDS-PAGE gel, transferred onto the PVDF membrane using a semi-dry protein transfer apparatus, and detected using the VECTASTAIN ABC-AmP detection system following the manufacturer's protocol. The intensities of protein bands are quantified with the ImageJ program (http://rsb.info.nih.gov/ij/) following the program manual.

While there has been described what is believed to be the preferred embodiments of the present invention, those skilled in the art will recognize that other and further changes and modifications may be made thereto without departing from the spirit of the invention. Therefore, the invention is not limited to the specific details and representative embodiments shown and described herein. Accordingly, persons skilled in this art will readily appreciate that various additional changes and modifications may be made without departing from the spirit or scope of the invention. In addition, the terminology and phraseology used herein is for purposes of description and should not be regarded as limiting. All documents referred herein are incorporated by reference into the present application as though fully set forth herein.

What is claimed is:

1. A method for labeling a protein comprising the steps of:
providing a synthetic small molecule tag;
providing a target protein to be tagged;
providing at least two enzymes for catalyzing a conjugation reaction between said tag and said target protein;
incubating said tag, said protein and said enzymes; and
allowing said tag to conjugate to said target protein.

2. The method set forth in claim 1, wherein said tag embodies at least one structural feature of an ubiquitin C-terminus.

3. The method set forth in claim 2, wherein said structural feature comprises a recognition sequence that is recognizable by an ubiquitin activating enzyme.

4. The method set forth in claim 1, wherein said tag comprises a probe and a recognition sequence.

5. The method set forth in claim 4, wherein said probe comprises biotin.

6. The method set forth in claim 4, wherein said recognition sequence is recognizable by an ubiquitin activating enzyme.

7. The method set forth in claim 4, wherein said probe and said recognition sequence are linked by a flexible aminohexanoic acid linker.

8. The method set forth in claim 4, wherein said probe is a fluorophore.

9. The method set forth in claim 1, wherein said target protein is a substrate for an ubiquitin conjugation system.

10. The method set forth in claim 1, wherein said target protein is tubulin.

11. The method set forth in claim 1, wherein said target protein is in the fraction two of a reticulocyte lysate.

12. The method set forth in claim 1, wherein said target protein is a ligase E3-specific substrate.

13. The method set forth in claim 1, wherein said enzymes are selected from a family of ubiquitin conjugating enzymes.

14. The method set forth in claim 1:
wherein said two enzymes are an ubiquitin activating enzyme and an ubiquitin conjugating enzyme;
further comprising the steps of providing an ubiquitin ligase enzyme;
incubating said tag, said protein, said activating enzyme and said conjugating enzyme; and
incubating said tag, said protein, said activating enzyme, said conjugating enzyme, and said ligase enzyme.

15. The method set forth in claim 1, wherein said step of incubating said tag, said protein and said enzymes comprises the step of applying an ATP-supplemented reaction buffer.

16. The method set forth in claim 1, wherein said step of incubating said tag, said protein and said enzymes comprises incubating at 37° C.

17. A compound for tagging a protein comprising:
a probe;
a recognition sequence that is recognizable by a ubiquitin activating enzyme.

18. The compound set forth in claim 17, wherein said recognition sequence is a peptide sequences derived from an ubiquitin C-terminus.

19. The compound set forth in claim 17, wherein said probe comprises biotin.

20. The compound set forth in claim 17, wherein said probe and said recognition sequence are linked by a flexible aminohexanoic acid linker.

21. The compound set forth in claim 17, wherein said probe is a fluorophore.

22. The compound set forth in claim 17, wherein said recognition sequence comprises a peptide sequence selected from a group consisting of Leu-Arg-Leu-Arg-Gly-Gly (SEQ ID NO: 1), Leu-Ala-Leu-Arg-Gly-Gly (SEQ ID NO: 2), Arg-Leu-Arg-Gly-Gly (SEQ ID NO: 3), Leu-Arg-Gly-Gly (SEQ ID NO: 4), Arg-Gly-Gly (SEQ ID NO: 5), and Gly-Gly (SEQ ID NO: 6).


SEQUENCE LISTING (6 sequences)

SEQ ID NO: 1 - protein, 6 aa, Artificial Sequence
  Designed peptide based on the C-terminus of ubiquitin
LRLRGG

SEQ ID NO: 2 - protein, 6 aa, Artificial Sequence
  Designed peptide based on C-terminus of ubiquitin tail
LALRGG

SEQ ID NO: 3 - protein, 5 aa, Artificial Sequence
  Designed peptide based on the C-terminus of ubiquitin
RLRGG

SEQ ID NO: 4 - protein, 4 aa, Artificial Sequence
  Designed peptide based on the C-terminus of ubiquitin
LRGG

SEQ ID NO: 5 - protein, 3 aa, Artificial Sequence
  Designed peptide based on the C-terminus of ubiquitin
RGG

SEQ ID NO: 6 - protein, 2 aa, Artificial Sequence
  Designed sequence based on the C-terminus of ubiquitin
GG